(19) United States
(12) Patent Application Publication
(10) Pub. No.: US 2004/0171008 A1
(43) Pub. Date: Sep. 2, 2004

(54) G-protein coupled receptor arrays

(76) Inventors: Kenneth Thirstrup, Kobenhavn (DK); Lars Siim Madsen, Kobenhavn (DK); Jens Bitsch Jensen, Kobenhavn (DK); Rene Hummel, Hellerup (DK); Bo Skaaning Jensen, Kobenhavn (DK)

Correspondence Address: BIRCH STEWART KOLASCH & BIRCH, PO BOX 747, FALLS CHURCH, VA, 22040-0747, US

(21) Appl. No.: 10/477,399

(22) PCT Filed: May 21, 2002
(86) PCT No.: PCT/DK02/00337

(30) Foreign Application Priority Data
May 18, 2001 (DE) PA 2001 00802

(51) Int. Cl. C12Q 1/68; C12M 1/34; C07K 14/705

(52) U.S. Cl. 435/6; 530/350; 435/287.2

(57) ABSTRACT

The invention relates to G-protein coupled receptor (GPCR) arrays, methods for production of GPCR arrays, primers used in the production of GPCR arrays and kits containing GPCR arrays and further to the use of such GPCR arrays in methods for the determination of expression profiles in biological materials in which there is an interest in the expression of GPCR polynucleotides.

### FIELD OF INVENTION

The invention relates to G-protein coupled receptor (GPCR) arrays, methods for production of GPCR arrays, primers used in the production of GPCR arrays and kits containing GPCR arrays and further to the use of such GPCR arrays in methods for the determination of expression profiles in biological materials in which there is an interest in the expression of GPCR polynucleotides.

### BACKGROUND OF INVENTION

Different kinds of arrays have become increasingly important tools in the biotechnology industry and related fields. These arrays have a plurality of polynucleotide spots deposited on a solid surface in form of an array. Arrays of both polypeptides and polynucleotides have been developed and find use in a variety of applications. One of the applications is differential gene expression, where expression of genes in different cells or tissues (normally a control sample and a sample of the cell or tissue of interest) is compared and any difference in the mRNA expression profile is determined.

In gene expression analysis using arrays, an array of “probe” nucleotides is contacted with a nucleic acid sample of interest such as mRNA concerted into cDNA from a particular tissue or cell. Contact is carried out under hybridisation conditions favourable for hybridisation of nucleic acids complementary to the “probe” nucleotides on the array. Unbound nucleic acid is then removed by washing. The resulting pattern of hybridised nucleic acid provides information regarding the gene expression profile of the sample tested on the array. Gene expression analysis is used in a variety of applications including identification of novel expression of genes, correlation of gene expression to a particular tissue or a particular disease, identifying effects of agents on the cellular expression such as in toxicity testing and in identifying drugs.

A variety of different array techniques have been developed during the years in order to meet the growing demands from the biotechnology industry, see e.g. Lockhart et al., Nature Biotechnology 1996 14 1675-1680; Shena et al., Science 1995 270 467-470 and WO 98/51789. However, there is still a need for new improved arrays having specific applications.

We hereby provide a novel array capable of determining the polynucleotide expression profile of GPCR derived polynucleotides in a biological material.

### BRIEF DISCLOSURE OF THE INVENTION

The object of the invention is to provide GPCR arrays, kits comprising GPCR arrays. and methods to produce such GPCR arrays. The GPCR arrays may be used for the determination of GPCR expression profiles in biological materials and also for the identification of therapeutic, prophylactic and/or toxic agents, where the therapeutic, prophylactic and/or toxic agents directly or indirectly influence the GPCR expression profiles in biological materials.

Accordingly, in a first aspect the invention relates to a GPCR array comprising a multiplicity of individual GPCR polynucleotide spots stably associated with a surface of a solid support, wherein an individual GPCR polynucleotide spot comprises a GPCR polynucleotide composition comprising a non-conserved region of a GPCR polynucleotide family member, the spots representing at least two different regions of a GPCR polynucleotide member of a family.

In another aspect, the invention relates to a method of preparing an array according to the invention, said method comprising generating said non-conserved regions of GPCR polynucleotide family members, preparing a multiplicity of compositions each comprising at least a non-conserved region, and stably associating said compositions in individual spots on a surface of a solid support.

In a further aspect, the invention relates to a set of primers specific for non-conserved regions of GPCR polynucleotide family members, wherein the set of primers are used in the method for the production of an array according to the invention.

In still a further aspect, the invention relates to a method for the determination of a GPCR polynucleotide expression profile in a biological material, said method comprising, obtaining a polynucleotide from the biological material, labelling said polynucleotide to obtain a labelled target polynucleotide sample, contacting at least one labelled target polynucleotide sample with an array according to the invention under conditions which are sufficient to produce a hybridisation pattern, and detecting said hybridisation pattern to obtain the GPCR polynucleotide expression profile of the biological material.

In still a further aspect, the invention relates to a method for the determination of a difference in GPCR polynucleotide expression profiles from at least a first and a second different biological materials, said method comprising obtaining a first GPCR expression profile of the first biological material according to the method of the present invention obtaining a second GPCR expression profile of the second biological material according to the method of the present invention, comparing the first and the second GPCR expression profile to identify any differences in the GPCR expression profiles between the first and the second GPCR expression profile.

In still a further aspect, the invention relates to a method for identifying a therapeutic, prophylactic and/or toxic agent involved in the response of GPCR polypeptides in a biological material, said method comprises obtaining a first GPCR expression profile of a first biological material according to the method of the present invention, obtaining a second GPCR expression profile of a second biological material according to the method of the present invention, treating the second biological material with a test compound; obtaining a third GPCR expression profile of the treated second biological material according to the method of the present invention, comparing the first, second and third GPCR expression profiles, and identifying any difference in the GPCR expression profile so as to identify any therapeutic, prophylactic or toxic response of the test compound on the GPCR polynucleotide in the second biological material.

In still a further aspect, the invention relates to a diagnostic method to determine the differences of GPCR expression profiles between two different biological materials; said method comprises obtaining a first GPCR expression profile of a first biological material according to the method of the present invention, obtaining a second GPCR expression profile of a second biological material according to the method of the present invention, comparing the first and second GPCR expression profiles, and identifying any difference in the GPCR expression profile.

In a final aspect, the invention relates to a GPCR kit for use in a hybridisation assay, said kit comprising a GPCR array according to the present invention.

The invention provides novel and improved GPCR arrays, kits comprising GPCR arrays and methods to produce such GPCR arrays. GPCR arrays are useful in the determination of GPCR expression profiles in biological materials and also in the identification of therapeutic, prophylactic and/or toxic agents; the therapeutic, prophylactic and/or toxic agent may directly or indirectly influence the GPCR expression profiles in biological materials.

### DETAILED DESCRIPTION OF THE INVENTION

The present invention provides a novel GPCR array. The GPCR array comprises e.g. a slide or a membrane or other kind of solid support onto which polynucleotide spots are applied and the polynucleotide spots represent one or more GPCR families or family members. Preferably, the polynucleotides (or fragments of polynucleotides) spotted on the slides have been chosen in such a way that the polynucleotides have specificity for more than one species such as e.g. humans, rats and mice, i.e. there is a certain interspecies identity, and at the same time, the polynucleotides chosen from a GPCR family member preferably have a certain degree of non-identity with other family members belonging to the same GPCR family, i.e. there is a relatively low intrafamily identity. By choosing the polynucleotides in such a manner, the use of a GPCR array of the invention makes it possible to obtain information relating to a GPCR family in general (and not only to a specific GPCR family member) and at the same time it is possible to compare and utilize information relevant for different species, cf. below.

Having a GPCR array containing polynucleotides spots with specificity for e.g. human, rat and mouse makes it possible to use the GPCR array on both human, rat and mouse derived biological material. It is therefore envisaged that the use of a GPCR array according to the present invention will lead to a better understanding of e.g. the pathogenesis of different ion related conditions or diseases in humans, since it is possible to compare biological material from humans with relevant biological material from e.g. well-known disease models in e.g. rats and mice.

If a GPCR is contemplated to be involved in the pathogenesis of a human related disease (e.g. as evidenced by the results of an analysis of RNA extracted from biopsies) the present invention makes it possible to confirm such a hypothesis by employing a GPCR array according to the invention. In such a case disease models in e.g. rats and mice are used and biological material obtained from e.g. diseased and healthy rats and/or mice is assayed by means of a GPCR array of the invention. In the same manner it is possible to confirm a hypothesis that a GPCR is involved in the pathogenesis in a disease model in e.g. rat or mouse (e.g. evidenced by analysis of RNA extracted from relevant tissue) also is associated with the condition in human. In such a case, RNA extracted from e.g. human biopsies is assayed by means of a GPCR array according to the invention and compared with the results from assays employing the relevant biological material from the diseased (and healthy) animals. Furthermore, it is possible to assess the expression level of the GPCR's across different tissues. In that way it is possible to address the question of the specificity the GPCR in the particular disease and gain information about the suitability of the GPCR as a target for the disease model.

As mentioned above, the spotted polynucleotides on the array of the invention have a certain cross-specificity to e.g. both human, rat and mouse. Samples from other species (e.g. pigs, dogs, chickens, cows and the like) can also be analysed on the GPCR array. This makes it possible to analyse tissue from other species with an unknown disease and compare it with tissue from well known e.g. neurological as well as other disease models in mice and rate. Such an approach leads to a better understanding of diseases in other species and initiates therapeutic strategies for e.g. economically expensive disease present in e.g. animal households.

Definitions

In the context of the present application and invention the following definitions apply:

The term “polynucleotide” is intended to mean a single or double stranded polymer composed of nucleotides, e.g. deoxyribonucleotides and/or ribonucleotides from about 30 to about 9,000 nucleotides in length, from about 50 to about 6,000, from about 50 to about 3,000, from about 50 to about 1,500, from about 50 to about 1,000, from about 100 to about 1,000, from about 200 to about 750, from about 200 to 700, from about 200 to 500 or from about 300 to about 350. The polynucleotides may be single or double stranded polynucleotides.

The term “complementary” or “complementarity“ is used in relation to the base-pairing rules of nucleotides well known for a person skilled in the art. Polynucleotides may be complete or partial complementary. Partial complementarity means that at least one nucleic acid base is not matched according to the base pairing rules. Complete complementarity means that all nucleotides in a polynucleotide match according to the base pairing rules. The degree of complementary between polynucleotides affects the strength of hybridisation between two polynucleotide strands. The inhibition by hybridisation of the complementary polynucleotide to the target polynucleotide may be analysed by techniques well known-for a person skilled in the art, such as Southern blot, Northern blot, and the like under conditions of high stringency. A partially (substantially) homologous polynucleotide will compete for and inhibit the binding of a completely homologous sequence to the target sequence under low stringency.

The term “homology” is intended to mean the degree of identity of one polynucleotide to another polynucleotide. According to the invention the term homology is used in connection with complementarity between polynucleotides within a family or between species. There may be complete homology (i.e. 100% identity) between two or more polynucleotides. The degree of homology may be determined by any method well known for a person skilled in the art.

The term “polynucleotide composition” is intended to mean a composition comprising a polynucleotide together with an excipient. The polynucleotide compositions are applied as spots on the array. The GPCR polynucleotide composition comprises a non-conserved region of a GPCR polynucleotide family member. The term “polynucleotide composition” includes also control or calibrating compositions such as, e.g. compositions comprising polynucleotides corresponding to housekeeping genes.

The term “non-conserved region” is intended to mean a segment of nucleotides in a polynucleotide, which compared to a segment of nucleotides in another polynucleotide has at the most about 90% identity. A non-conserved region of a GPCR polynucleotide family member is thus defined as a region of nucleotides corresponding to part of the polynucleotide, and the non-conserved region has less than 90% such as, e.g. less than 85% less than about 80%, less than about 75% or less than about 70% identity compared to all other polynucleotides belonging to the same GPCR polynucleotide family (intrafamily identity). In particular the non-conserved regions will be found in polynucleotides corresponding to the intra- or extra cellular loops or the N- or C-terminal part of GPCR.

Accordingly, the term “conserved region” is intended to mean a segment of nucleotides in a polynucleotide, which compared to a segment of nucleotides in another polynucleotide has more than 90%, such as at least about 92%, at least about 95% or at least about 97% identity.

The term “GPCR” is intended to mean a polypeptide which is a transmembrane protein consisting of seven transmembrane domains with same overall structure. The majority of the GPCR's have the ability to transduce a signal across the cell membrane through activation of a G-protein on the intracellular side of the cell. However, some of these receptors may also be signalling via alternative signal molecules like Jak2 kinases, phospholipase Cγ or protein kinase C.

The GPCR's are classified upon their ligand specificity, mode of activation, biological function, regulation or molecular structure. More than 2000 G protein coupled receptors have been reported since the cloning of bovine opsin receptor in 1983 and they have been classified into over 100 subfamilies according to sequence homology, ligand structure, and receptor function. Examples of GPCR's are adrenergic, adenosine, dopaminergic, histaminergic, opioid and serotonin GPCR's.

GPCR's are direct or indirect targets for the action of compounds, such as drugs.

The term “GPCR polynucleotide” is intended to mean a polynucleotide encoding a polypeptide (GPCR) involved in transducing a signal across biological membranes.

The term “GPCR family” is intended to mean a group of GPCR polypeptides, which has common characteristics such as, e.g. ligand specificity (i.e. the ability to bind a ligand with almost similar affinity and efficacy), homology, and same overall tertiary amino acid structure. Each family comprises individual members each having structural variations but they fulfil the requirements mentioned above with respect to being classified as a family. An example of a GPCR subfamily is the opioid receptor family consisting of the μ, δ and κ-opioid receptors (see also Example 1).

The term “GPCR polynucleotide family” is intended to mean polynucleotides encoding polypeptides of a GPCR family”. The polynucleotides may generally be found and downloaded from Genbank or EMBL (see e.g. http:www.ncbi.nih.org).

The term “intrafamily identity” is intended to mean identity within a group of members belonging to the same level in the taxonomic system, e.g. superfamily, family or subfamily or a number of subfamilies. The level in the taxonomic system to be used is selected depending i.a. on the number of members in the level. Preferably, the level in the taxonomic system is selected so that the number of members is from 2-20, more preferably 3-15, more preferably 4-12, and most preferably 5-10.

The term “interspecies identity” is intended to mean identity between a group of different species, such as a group comprising humans, mice and rats.

The terms “expression profile”, “differential expression profile” and “gene expression profile” are intended to mean the expression of the mRNA's in a biological material. While an expression profile encompasses a representation of the expression level of at least one mRNA, in practice the typical expression profile represents the expression of several mRNA's. For example, an expression profile used according to the present invention represents the expression levels of at least from about 1 to 50,000 or more different mRNA's in a biological material. The expression level of the different m RNA's is the same or different. The expression of mRNA's may be up- or down regulated resulting in different expression profiles.

The term “biological material” includes within its meaning organisms, organs, tissues, cells or biological material produced by a cell culture. The biological material may be living or dead. The material may correspond to one or more cells from the organisms, in case the organism is a multicellular organism, the material may correspond to one or more cells from one or more tissues creating the multicellular organism. The biological material to be used according to the invention may be derived from particular organs or tissues of the multicellular organism, or from isolated cells obtained from a single or multicellular organism. In obtaining the sample of RNA's to be analysed from the biological material from which it is derived, the biological material may be subject to a number of different processing steps. Such steps might include issue homogenisation, cell isolation and cytoplasma extraction, nucleic acid extraction and the like and such processing steps are generally well known for a person skilled in the art. Methods of isolating RNA from cells, tissues, organs or whole organisms are known to those skilled in the art and are described in e.g. Sambrook et al, Molecular Cloning: A Laboratory Manual, Cold Spring Harbour Press (1989). The biological material may be of the same kind i.e. the biological material is of the same kind of origin, such as coming from the same type of tissue, he same organism or the same type of organism or the same cell type etc.

The term “organism” is intended to mean any single cell organism such as yeast or multicellular organism, including plants, fungi and animals, preferably mammals, such as humans, rats, pigs, cows, horses, dogs, guinea pigs, ferrets, rabbits, sheep, apes, monkeys and cats.

The term “tissue” is intended to mean a collection of differentiated cells such as adrenal gland, total brain, liver, heart, kidney, lung, pancreas, mammary gland, placenta, prostate, salivary gland, skeletal muscle, small intestine, spleen, stomach, testis, thymus, trachea, and uterus.

The term “target polynucleotide” is intended to mean a polynucleotide present in the biological material of interest. The target polynucleotide encodes a polypeptide, which is at least a part of a GPCR. If the target polynucleotide has a complementary polynucleotide present on the GPCR array, it will hybridise thereto and thus give rise to a detectable signal.

The term “non-overlapping” is intended to mean that when the GPCR polynucleotide regions used in the GPCR polynucleotide composition spots are obtained from the same polynucleotide, the regions are obtained from different parts of the polynucleotide and the different parts are located in such a manner that the regions not even overlap each other by a single nucleotide. In a polynucleotide of e.g. 1,000 nucleotides the regions 1-500 and 501-900 are non-overlapping. The non-overlapping GPCR polynucleotide regions may be located with a distance of one or more nucleotides from each other.

The term “primer” is intended to mean a polymer of 10-50 nucleotides.

The term “set of primers” is intended to mean one or more primers having the ability to amplify a GPCR polynucleotide region under suitable conditions. The length of the primers may be the same or different and dependent on the character of the GPCR polynucleotide region to be amplified. Design of such a set of primers is well known for a person skilled in the art. The set of primers having a sufficient length to specifically hybridise to a distinct GPCR polynucleotide in the sample and the length of the primers will be from about 3 to 50 nucleotides.

The terms “stressed state” and “stressed” are intended to mean that the above described “biological material” is influenced compared to the normal condition. When an expression profile is obtained from a stressed biological material it is different compared to a non-stressed biological material. The biological material may be influenced by some kind of organic/inorganic compound, an environmental agent, a drug substance, pathogen, mutagen, mitogen, receptor mediated signal or the like. Normally, the biological material is influenced in such a manner that the expression profile of the GPCR polynucleotides in the biological material either directly or indirectly is affected resulting in at least one difference between the expression profile of the non-stressed biological material compared to the stressed biological material.

Methods of comparing the homology between different polynucleotides and/or parts of different polynucleotides irrespective of whether the parts are conserved or non-conserved regions are well known in the art. The polynucleotides may either belong to the same family or different families and/or being polynucleotides encoding the same polypeptide from the same or different species. Optimal alignment of nucleotides of a polynucleotide for comparison of the homologies may be conducted using the homology algorithm described by e.g. Smith and Waterman, Adv. Appl. Math. 1981 2 482, by the homology alignment algorithm of Needleman and Wunsch, J. Mol. Biol. 1970 48 443, by the search for similarity method of Pearson and Lipman, Proc. Natl. Acad. Sci. USA 1988 85 2444, or by computerised implementations of these algorithms by using for example CLUSTAL in the PC/Gene program by Intelligenetics, Mountain View, Calif., GAP, BESTFIT, FASTA, and TFASTA in the Wisconsin Genetics Software Package, Genetics Computer Group (GCG) 575 Science Dr., Madison, Wis., USA. The above-mentioned algorithms and computer implementations should be regarded as examples and the invention not limited thereto.

GPCRs

It has been estimated that up to 5000 distinct GPCR-encoding genes exist within the human genome. Up to now approximately 800 genes have been cloned from various species, including approximately 150 human genes. The GPCR superfamily may be classified in three major homology families for the mammalian GPCRs, viz. the family 1 or rho-family (prototype: rhodopsin), the family 2 or scr-family (prototype: secretin receptor), and the family 3 or mGluR family (prototype: metabotropic glutamate receptors.

Family 1 is divided according to the size and chemical nature of the corresponding agonists, as well as the mode of ligand binding. Family 1a accommodates the β-adrenoceptor-type receptors that are activated by small ligands, such as biogenic monoamines, opiates, nucleotides and small peptides that bind to a transmembrane cavity formed by helices 3, 4, 5 and 6. Family 1b is composed of receptors stimulated by oligopeptides and proteins, such as IL-8 (interleukin-8), cytokines and thrombin. The ligand binding epitope is located in the extracellular loop region. Family 1c receptors recognise glycoprotien hormones, such as LH (luteinising hormone), TSH (thyroid-stimulating hormone), and FSH (follicle-stimulating hormone), while their ligand binding site is centered in a large extracellular N-terminal domain.

Family 2 receptors are distinct from the rho-family receptors in that they bind large molecules like glucagons, secretin, PTH (parathyroid hormone), VIP (vasointestinal peptide), or CRF (corticotropin-releasing factor). Comparable to family 1c receptors, the secretin family utilises a large N-terminal domain for ligand binding. Family 3 receptors are unique in that they possess a large extracellular N-terminal domain of several hundred residues that constitutes the binding site for smallish ligands, such as a single divalent Ca²⁺ cation, glutamate, GABA (γ-amino butyric acid) and pheromones.

Examples of GPCR members and GPCR subfamilies are given in the following Table 1:

TABLE 1

| | | Native ligand | Nature of the |
|---|---|---|---|
| GPCR | code | (peptide/protein) | ligand |
| angiotensin receptors | AT₁, AT₂ | angiotensin II (All) | Asp-Arg-Val-Tyr-Ile |
| | | | His-Pro-Phe |
| bombesin receptors | BB1-BB4 | bombesin, | 14 aa peptide amide |
| | | neuromedin B, | |
| | | gastrin-releasing | |
| | | peptide | |
| bradykinin receptors | B₁, B₂ | bradykinin (BK) | Arg-Pro-Pro-Gly-Phe- |
| | | | Ser-Pro-Phe-Arg |
| C3a receptor | C3aR | C3a anaphylatoxin | protein |
| C5a receptor | C5aR | C5a anaphylatoxin | protein |
| CC chemokine | CCR1-CCR9 | chemokines | proteins |
| receptors | | | |
| CXC chemokine | CXCR1-CXCR5 | chemokines | proteins |
| receptors | | | |
| cholecystokinin/gastrin | CCK_{A}, CCK_{B} | cholecystokinin | 33 aa peptide amide, |
| receptors | | (CCK), gastrin | 17 aa peptide amide |
| endothelin receptors | ET_{A}-ET_{B} | endothelin-1 (ET-1), | 21 aa peptides |
| | | ET-2, ET-3 | |
| alpha factor | STE2, STE3 | fungal mating | 13 aa peptide |
| pheromone receptor | | pheromones | |
| fMet-Leu-Phe receptor | fMLP-R | Formylpeptide (fMLP) | FMet-Leu-Phe |
| galanin | GAL1, gal2, gal3 | galanin | 30 aa peptide |
| melanocortin | MC₁, MC₃, MC₄, MC₅ | melanocortin (MSH) | 39 aa peptide |
| receptors & ACTH | MC₂ = ACTH | adrenocorticotropic | |
| receptor | receptor | hormone (ACTH), | |
| | | corticotropin | |
| neuropeptide Y | Y₁-Y₆ | neuropeptide Y | 36 aa peptide amide |
| receptor | | (NPY), peptide YY | (NPY) |
| | | (PYY), pancreatic | |
| | | polypeptide (PP) | |
| neurotensin receptor | NTS1, nts2 | neurotensin | 13 aa peptide |
| opioid receptors | δ | [Met]-enkephalin, | Tyr-Gly-Gly-Phe- |
| | | [Leu]-enkephalin | Met/Leu |
| | κ | dynorphin A | 17 aa peptide |
| | μ | β-endorphin, | 31 aa peptide |
| | | Lipotropin C fragment | |
| nociceptin receptor | ORL1 | nociceptin, orphanin | 17 aa peptide |
| | | FQ | |
| somatostatin | sst₁-sst₅ | somatostatin | cyclic 14 aa peptide |
| receptors | | | |
| tachykinin receptors | NK₁ | substance P | 11 aa peptide |
| | NK₂ | neurokinin A (NKA), | His-Lys-Thr-Asp-Ser- |
| | | substance K, | Phe-Val-Gly-Leu- |
| | | neuromedin L | Met-NH₂ |
| | NK₃ | neurokinin B (NKB), | Asp-Met-His-Asp- |
| | | neuromedin K | Phe-Phe-Val-Gly- |
| | | | Leu-Met-NH₂ |
| thrombin/protease- | PAR1, PAR2, | thrombin, trypsin, | protein |
| activated receptors | PAR3, PAR4 | factor Xa | |
| vasopressin receptors | V_{1A}, V_{1B}, V₂ | vasopressin | Cys-Tyr-Phe-Gln- |
| | | | Asn-Cys-Pro-Arg- |
| | | | Gly-NH₂ |
| oxytocin receptor | OT | oxytocin | Cys-Tyr-Ile-Gln-Asn- |
| | | | Cys-Pro-Leu-Gly-NH₂ |
| vasotocin receptor | VT | vasotocin | Cys-Tyr-Ile-Gln-Asn- |
| | | | Cys-Pro-Arg-Gly-NH₂ |
| orexin receptors | OX₁, OX₂ | orexin A/B | 33 aa/28 aa peptide |
| | | | amides |
| FSH receptor | FSH receptor | follicle-stimulating | protein |
| | | hormone (FSH) | |
| LSH receptor | LSH receptor | lutropin, | protein |
| | | choriogonadotropic | |
| | | hormone, luteinizing | |
| | | hormone | |
| TSH receptors | TSH receptor | thyrotropin, thyroid- | protein |
| | | stimulating hormone | |
| LHRH receptor | LHRH receptor | gonadotropin- | pGlu-His-Trp-Ser- |
| | | releasing hormone | Tyr-Gly-Leu-Arg-Pro- |
| | | (GnRH), luteinizing | Gly-NH₂ |
| | | hormone-releasing | |
| | | hormone (LHRH) | |
| thyrotropin-releasing | TRH₁, trh₂ | thyrotropin-releasing | pGlu-His-Pro-NH₂ |
| hormone & | | hormone/factor | |
| secretagogue | | (TRH/F) | |
| receptors | | | |
| GHS receptor | GHSR₁ₐ, GHSR_{1b} | growth hormone | oligopeptides |
| | | secretagogues (GHS) | |
| calcitonin/calcitonin | CGRPR | calcitonin, calcitonin | 32 aa peptide amide |
| gene-related peptide | | gene-related peptide | |
| receptors | | (CGRP) | |
| amylin receptor | amylin receptor | amylin | 37 aa peptide amide |
| adrenomedullin | adrenomedullin | adrenomedullin | 52 aa peptide amide |
| receptor | receptor | | |
| corticotropin-releasing | CRF₁, CRF₂ | corticotropin- | 41 aa peptide amide |
| factor receptor | | releasing factor | |
| | | (CRF) | |
| gastric inhibitory | gip receptor | gastric inhibitory | 42 aa peptide |
| peptide receptor | | peptide (GIP) | |
| glucagon/glucagon- | GLP1 | glucagon | 29 aa peptide |
| like peptide receptor | | | |
| growth-hormone- | GHRH receptor | growth-homone- | 44 aa peptide amide |
| releasing hormone | | releasing | |
| receptor | | homone/factor | |
| | | (GHRH/GRF) | |
| parathyroid hormone | type 1, type 2 | parathyroid hormone | 84 aa peptide |
| receptor | | (PTH) | |
| secretin receptor | secretin receptor | secretin | 27 aa peptide amide |
| vasoactive intestinal | VPAC₁, VPAC₂, | vasoactive intestinal | 28 aa peptide amide |
| peptide & PACAP | PAC₁ | peptide (VIP) pituitary | 38 aa peptide |
| receptor | | adenylate cyclase | |
| | | activating peptide | |
| | | (PACAP) | |

GPCR Arrays

A GPCR array according to the invention has a multiplicity of individual GPCR polynucleotide spots, stably associated with a surface of a solid support. Each spot on the GPCR array comprises a GPCR polynucleotide composition, wherein the polynucleotide regions within the composition are of known identity, usually of known sequence, as described later on in detail. The GPCR polynucleotide spots may be of convenient shape but most often circular, oval or any other suitable shape. The GPCR polynucleotide spots may be arranged in any convenient pattern across the surface of the solid support, such as in row or columns to form a grid, in a circular pattern and the like. Preferably the pattern of GPCR polynucleotide spots is arranged as a grid to facilitate the evaluation of the results obtained from the analyses in which the. GPCR array is used.

The GPCR array according to the invention may be of a flexible or rigid solid support and the GPCR polynucleotide spots are stably associated thereto. By stably associated is meant that the GPCR polynucleotide spots will be associated in their position on the solid support during the analysis in which the GPCR array is used, such as during different hybridisation, washing and detection conditions. The GPCR polynucleotide regions contained in the spots may be covalently or non-covalent associated to the surface of the solid support. Methods how to covalently or non-covalently bind the GPCR polynucleotide regions to the surface of the solid support are well known for a person skilled in the art and may be found in e.g. Ausubel et al., Current Protocols in Molecular Biology, Greene Publishing Co. NY, (1995).

The solid support to which the individual GPCR polynucleotide spots are stably associated to is made of a flexible or rigid material. By flexible is meant that the support is capable of being bent or folded without breakage. By rigid is meant that the support is solid and does not readily bend, i.e. the support is not flexible. The support may be fabricated from a variety of materials, including plastics, ceramics, metals, gels, nitrocellulose, nylon, glass and the like.

The array may be produced according to any convenient methodology, such as preparing or obtaining the polynucleotides and then stably associate them with the surface of the support or growing them directly on the support. A number of array configurations and methods for their production are known to those skilled in the art and disclosed in e.g. U.S. Pat. No. 5,445,934, U.S. Pat. No. 5,532,128, U.S. Pat. No. 5,556,752, U.S. Pat. No. 5,242,974 U.S. Pat. No. 5,384,261, U.S. Pat. No. 5,405,783, U.S. Pat. No. 5,412,087, U.S. Pat. No. 5,424,186, U.S. U.S. Pat. No. 5,429,807 U.S. Pat. No. 5,436,327, U.S. Pat. No. 5,472,672 U.S. Pat. No. 5,527,681 U.S. Pat. No. 5,529,756 U.S. Pat. No. 5,545,531 U.S. Pat. No. 5,554,501 U.S. Pat. No. 5,561,071, U.S. Pat. No. 5,571,639 U.S. Pat. No. 5,593,839 U.S. Pat. No. 5,599,659 U.S. Pat. No. 5,624,711 U.S. Pat. No. 5,658,734 and U.S. Pat. No. 5,700,637.

The solid support of the invention may have several configurations ranging from a simple to a more complex configuration depending on the intended use of the GPCR array. The size and thickness of the GPCR array is not critical as long as the GPCR array will function in the expected way and as long as the results obtained after use of the GPCR array are not changed. The number and amount of the GPCR polynucleotide spots is dependent on the intended use of the GPCR arrays as well as the detection system use to determine the expression profile of the biological material being evaluated by the aid of the GPCR array. The number of the GPCR polynucleotide spots may vary from about 2 to about 100,000 such as e.g. from about 2 to about 50,000, from about 10 to about 25,000, from about 100 to about 10,000, from about 100 to about 5,000, from about 100 to about 1,000, from about 400. to about 600 or about 500 GPCR polynucleotide spots, or at least 2 such as, e.g. at least 10, at least 25, at least 50, at least 100, at least 300, at least 400, at least 500 or at least 600 spots, or even more than 100,000 spots. The limitations of the number of the GPCR polynucleotide spots are dependent on the way in which the evaluation of the expression profile of the biological material is performed. The amount of the GPCR polynucleotide regions present in the GPCR polynucleotide spot may vary and the amount will be sufficient to provide adequate hybridisation and detection of the target nucleic acid. Generally the GPCR polynucleotides will be present in each spot at a concentration corresponding to an amount of 1 ng-10 μg per μl or less than 1 μg of the polynucleotide. Normally, only 1 GPCR polynucleotide region is present in each spot.

The copy number of the GPCR polynucleotide present in each GPCR polynucleotide spot will be sufficient to provide enough hybridisabon for a target nucleic acid to yield a detectable signal, and generally range from about 50 fmol or less.

An important feature of the GPCR array is i) that the majority of the GPCR polynucleotide spots represent GPCR families which have GPCR members, ii) the GPCR polynucleotide regions present in the GPCR polynucleotide spots are made up from non-conserved regions of the GPCR family members, and iii) at least two GPCR polynucleotide regions representing each GPCR family member are present on the GPCR array. The two or more GPCR polynucleotide regions from one GPCR family member are chosen in such a way that they are non-overlapping regions. The use of two or more GPCR polynucleotide regions on the GPCR array ensures a proper expression profile from the same GPCR polynucleotide. In general, prior art arrays have suffered from the technical problem that they are not fully reliable in the sense that they produce a certain level of both false negative and false positive results. This technical problem has been solved with the present invention by the use of at least two regions of the same GPCR polynucleotide family member. Such array design greatly increases the reliability of the results generated by the array, as each determination has at least one double check or verification (positive and negative control). Also, this double check may be effected in a controlled manner and to a pre-determined level by selection of an appropriate number of regions of a GPCR polynucleotide family member. The level of double check, i.e. the number of regions, may be selected specifically for the intended use and depends on several factors, which includes, but are not limited to, the length of the polynucleotide regions spotted, the degree of intra-family identity, the degree of interspecies identity, the array hybridisation conditions, the characteristics of the biological polynucleotide test sample etc.

The number of non-conserved polypeptide regions to be chosen is dependent i.a. on the length of the corresponding GPCR polynucleotide. Additionally, the ability to identify expression of GPCR polynucleotide which for some reason have a mutation or a deletion may increase by the use of more than one non-conserved polynucleotide region from each member-of a GPCR family. Generally the non-conserved regions will be found in the three extracellular loops, the three intracellular loops or in the N- or C-terminal end of the GPCR.

Preferably, each type of spot as defined by its content of polynucleotide region is present in a number of copies, such as 2, 3, 4, 5 or 6, in order to enhance the reliability of the results obtained in the use of the array. When multiple spots of the same type are used, the mean value of the results obtained is calculated and used.

The array according to the invention has several different regions of a GPCR polynucleotide family member, which may be polynucleotide regions from the same polynucleotide strand and the regions differ at least by one nucleotide.

The non-conserved regions corresponding to a specific GPCR member of a GPCR family are preferably selected such that the selected GPCR regions have the ability to hybridise to the corresponding polynucleotide from more than one species. One selected GPCR region may be used for the identification of the expression profile of a certain GPCR polynucleotide in several biological materials obtained from several species such as e.g. humans, mice and rats. By this feature the functionality of the GPCR array increases such that solely one type of GPCR array is needed for the evaluation of the expression profiles of GPCR polynucleotide in biological materials obtained from several species. One GPCR polynucleotide spot will hybridise specifically to one single member of a GPCR family due to the selected non-conserved region of that particular GPCR. By the use of such a strategy for the development of the GPCR array, several different biological materials such as, e.g. material obtained from different species of animals may be used and compared for their expression profile of GPCR polynucleotides using only one type of GPCR array. The same strategy also applies for plants, fungi, microorganisms etc.

Other polynucleotide spots (control spots), which may be present on the GPCR array, include spots comprising genomic DNA, housekeeping genes, negative and positive control polynucleotides and the like. These polynucleotide spots comprise polynucleotides, which are not unique, i.e. they are not polynucleotide regions corresponding to GPCR polynucleotides. They are used for calibration or as control polynucleotides, and the function of these polynucleotide spots are not to give information of the expression of these polynucleotides, but rather to provide useful information, such as background or basal level of expression to verify that the analysis and the expression profiles obtained are relevant or not. Furthermore these control spots may serve as orientation spots.

The GPCR polynucleotides of interest in the present context are those, which encode seven transmembrane polypeptides involved in transducing a signal across a biological membrane. Examples of suitable GPCR polynucleotide families are adrenergic, adenosine, dopaminergic, histaminergic, opioid and serotonin GPCR's and any other seven transmembrane polynucleotides capable of transducing a signal across biological membranes.

The GPCR polynucleotides to be stably associated to the solid support may be of DNA, RNA, cDNA, natural, synthetic, semi-synthetic origin or chemical analogous such as LNA or PNA. The GPCR polynucleotides may be obtained from one or more biological material such as an organism, an organ, a tissue and/or a cell and/or produced by a cell culture. The biological material may be obtained from any kind of organism, such as a microorganism, a plant, a fungus (e.g. yeast, mushrooms), animal or tissue. Examples of animals from which one or more biological material may be obtained are humans, rats, mice, pigs, cows, horses, dogs, guinea pigs, ferrets, rabbits, apes, monkeys, cats and sheep. The GPCR polypeptides involved in transducing a signal across biological membranes may be located e.g. in an organ such as heart, liver, prostate, brain, kidney, lung etc., tissue such as nerve, muscle, connective, etc., and/or they may be found in the cells such as e.g. in the nucleus, endoplasmatic reticulum, Golgi complex, endosome, lysosome, peroxisome, mitochondria, cytoplasm, plasma membrane, cytoskeleton.

The length of the GPCR polynucleotides present in the GPCR polynucleotide spot is selected in such a manner that the length is sufficient to provide a strong, specific and reproducible signal. The length will typically vary from about 3 to about 9,000 nucleotides such as e.g. from about 3 to about 6,000, from about 3 to about 3,000, from about 10 to about 1,500, from about 50 to about 1,000, from about 100 to about 800, from about 200 to 750, from about 200 to 700, from about 200 to 500, from about 250 to 400 or preferably from about 300 to 350. However, the length of the GPCR polynucleotides present on the GPCR array is shorter than the length of the mRNA to which it corresponds. As such, the GPCR polynucleotide represents a part of the full-length cDNA to which it corresponds. The length of the GPCR polynucleotide region present in the GPCR spot is dependent on the number of polynucleotides in the selected GPCR family member.

The non-conserved regions of GPCR polynucleotide regions contained in a GPCR polynucleotide composition may be single or double stranded non-conserved polynucleotide regions.

The GPCR polynucleotide composition also comprises an excipient. Suitable excipients are solvents like e.g. water or any other aqueous medium, pH adjusting agents like buffering agents, stabilising agents, hybridising agents, colouring agents, labelling agents and the like. In general,.the excipients used are inert, i.e. they do not have any polynucleotide related effect.

Method of Selecting the GPCR Polynucleotides

According to the present invention an important feature of the GPCR array is that the majority of the GPCR polynucleotide spots are made up from family members of GPCR polynucleotides and the GPCR polynucleotide regions present in the GPCR polynucleotide spots are made up from non-conserved regions of the GPCR polynucleotides. The sequences of the GPCR families may be found in GenBank (http.www.ncbi.nih.gov) and downloaded prior to sequence comparison. The sequence comparisons may be performed using any of the methods mentioned above. An example is given in Example 1 herein.

Additionally, the GPCR array preferably represents at least two different GPCR polynucleotide members of a family and/or at least two different GPCR families.

Furthermore, at least two of the GPCR polynucleotide spots are made up from one GPCR polynucleotide of one GPCR polynucleotide family member. The GPCR polynucleotide regions present in the two GPCR spots are made up from regions of one and the same GPCR polynucleotide and the regions are at least non-overlapping with a distance of at least one nucleotide from each other. The GPCR polynucleotide regions are selected in such a way that they are non-conserved regions within the same GPCR family member (intrafamily) and the regions have at least 50% identity between different species (interspecies). The strategy how to find and identify potential GPCR regions useful to stably associate onto the surface of the GPCR array will be described in detail hereinafter.

In one embodiment of the invention relates to a GPCR array comprising a multiplicity of individual GPCR polynucleotide spots stably associated with a surface of a solid support, wherein an individual GPCR polynucleotide spot comprises a GPCR polynucleotide composition, and the spots represent at least two different regions of a GPCR polynucleotide family member. The GPCR polynucleotide composition comprises a non-conserved region of a GPCR polynucleotide family member. The non-conserved regions of a GPCR polynucleotide family member is a stretch of nucleotides with an average length of from about 3 to about 9,000 nucleotides such as, e.g. from about 3 to about 6,000, from about 3 to about 3,000, from about 5 to 1,500, from about 10 to about 1,000, from about 50 to about 1,000, from about 100 to about 1,000, from about 200 to 750, from about 200 to 700, from about 200 to 500, from about 250 to 400 or from about 300 to 350. The non-conserved regions of a GPCR polynucleotide family member is a region of nucleotides which has less than 90% such as, e.g. less than 85%, less than about 80%, less than about 75%, less than about 70%, less than about 65%, less than about 60%, less than about 55% or less than about 50% intrafamily identity, which means less than 90% (or, alternatively 85%, 80%, 75% or 70%) identity between polynucleotides classified as member of a specific GPCR family, see example 1. The homology between members of a certain GPCR family may be determined using the methods mentioned above.

The two or more different non-conserved polypeptide regions corresponding to one GPCR member may be identified using the same strategy and hey may at least be non-overlapping regions as mentioned above.

The non-overlapping regions may be selected from just one non-conserved region in case the polynucleotide GPCR family member contains just one non-conserved region.

The non-conserved polypeptide regions may furthermore be selected on the basis of homology of specific regions between different species (interspecies), such as between species of microorganisms, fungi, plants or animals such as e.g. humans, rats, mice, pigs, cows, horses, dogs, guinea pigs, ferrets, rabbits, apes, monkeys, cats and sheep.

The non-conserved region of the GPCR family member may have at least 50% interspecies identity such as, e.g. at least about 60%, at least about 65%, at least about 70%, at least about 75% or at least 80% interspecies identity. By the use of such as strategy in which the non-conserved regions are selected on the basis of their interspecies identity, it is possible to use the same type of GPCR array for detection of GPCR expression profiles in several different species as long as the selected non-conserved regions shares such a high degree of homology to enable hybridisation.

The difference between the percent interspecies identity and the percent intrafamily identity (percent interspecies identity minus percent intrafamily) should be a least 5%, at least 10%, at least 15%, at least 20% and at least 25%. By such a strategy it should be possible to select hybridisation conditions, which may preferentially bind interspecies related polynucleotides and avoid the binding of intrafamily related polynucleotides.

The non-conserved region of the GPCR polynucleotide used according to the invention will generally be a single stranded polynucleotide and shorter than the mRNA to which it corresponds.

In one embodiment of the array of the invention, at least one non-conserved polynucleotide region is present in the form of sense single-strands in a spot. In conventional arrays the polynucleotides are usually present in double-stranded form, which is denatured by heating prior to contacting with the biological sample to make the sense strand available for binding with the sample polynucleotides. It is believed that in such conventional arrays a certain variable and non-controlled level of the double-stranded polynucleotides on the array does not in fact separate sufficiently to allow hybridisation with the sample polynucleotide. In comparison, an array wherein the sense polynucleotide is present in a single-stranded form has the advantage that all strands are available for hybridisation thus resulting in an increased and more reproducible level of binding and array response. Preferably, he said non-conserved polynucleotide region present in the form of sense single-stranded in a spot is also present in the form of antisense single-strands in a separate pot. This embodiment involves the advantage that the spot containing the antsense strands serves as a negative control for a positive determination in the spot containing the corresponding sense strands. Thus, this embodiment of the invention significantly increases the reliability of the results obtained in the use of the array. Furthermore, when only the sense sequence of the polynucleotide region is included in the array, it is necessary to identify the sense strand of the double-stranded polynucleotide region during the preparation of the array. In comparison, when both the sense and the anti-sense strand of the polynucleotide region are included as separate spots, identification of which is which is avoided hence facilitating the preparation of the array, which then can be carried out using standard methods.

When the array is to be used for screening biological polynucleotide samples originating exclusively or primarily from one animal species, the GPCR polynucleotide regions present in the spots preferably originate from the same species. This is preferred because it will give an optimum level of identity between the polynucleotides of the spot on the one side and the polynucleotides of the biological material on the other side, and hence a more reliable determination.

Method of Preparing a GPCR Array

The GPCR array may be prepared (produced) using any convenient method and several methods are well known for a person skilled in the art, such as standard procedures according to e.g. Sambrook et al, Molecular cloning: A laboratory manual 2^{nd}edition, Cold Spring Harbour Laboratory Press, New York.

One means of preparing the GPCR array is i) synthesising or otherwise obtaining the above mentioned non-conserved GPCR polynucleotide regions, ii) preparing the GPCR polynucleotide compositions to be used in each spot and then iii) depositing in the form of spots the polynucleotide compositions comprising the non-conserved GPCR polynucleotide regions onto the surface of the solid support, see also Examples 2-5. The non-conserved GPCR polynucleotide regions may be of DNA, RNA, cDNA, natural, synthetic, semi-synthetic origin or chemical analogous such as LNA or PNA. The non-conserved regions may be obtained from any biological material such as e.g. tissues or cells and/or produced by a cell culture. The biological material may be an organism, such as a microorganism, plant, fungus (e.g. yeast or mushrooms) or animal. If the organism is an animal it may be selected from a group consisting of humans, rats, mice, pigs, cows, horses, dogs, guinea pigs, ferrets, rabbits or sheep.

The non-conserved GPCR polynucleotide regions may be prepared using any conventional methodology such as automated solid phase synthesis protocols, PCR using one or more primers specific for the non-conserved GPCR polynucleotide regions and the like. In general, PCR is advantageous in view of the large numbers of non-conserved GPCR polynucleotide regions that must be generated for each GPCR array. The amplified non-conserved GPCR polynucleotide regions may further be cloned in any suitable plasmid vector to enable multiplication and storage of the amplified non-conserved GPCR polynucleotide regions (see Examples 3-4).

The prepared non-conserved GPCR polynucleotide regions may be spotted onto the solid support using any convenient methodology, including manual and automated techniques, e.g. by micro-pipette, ink jet pins etc. and any other suitable automated systems. An example of an automated system is the automated spotting device Beckman Biomek 2000 (Beckman Instruments, USA). The ready GPCR arrays may then be stored at suitable conditions until use.

Method for the Determination of GPCR Expression Profiles

Determination of GPCR expression profiles typically means determination of the expression level of multiple mRNA's, all of them corresponding to GPCR polynucleotides. The detection limit of the expression level of mRNA may be approximately 0.2 ng or less of total RNA of the biological material used to hybridise each individual GPCR polynucleotide spot.

The expression profiles can be produced by any means known in the art, including but not limited to the methods disclosed by e.g. Liang et al., Science 1992 257 967-971; Ivanova et al., Nucleic Acids Res. 1995 23 2954-2958; Guilfoyl et al., Nucleic Acids Res. 1997 25 (9) 1854-1858; Chee et al, Science 1996 274 610-614; Velculescu et al., Science 1995 270 484-487; Fiscker et al., Proc. Natl. Acad. Sci. USA 1995 92 (12) 5331-5335; and Kato, Nucleic Acids Res. 1995 23 (18) 3685-3690.

The hybridisation conditions under which the biological polynucleotide sample is contacted with the array of the invention may vary and are selected to suit the characteristics of the specific array/sample system as well as the purpose of the use of the array. The hybridisation conditions selected depend e.g. on the species from which the biological sample originates, the length of the polynucleotide regions in the spots, the number of polynucleotide regions from the same GPCR family member, the level of intra-family identity, the level of interspecies identity, the array reaction conditions, such as the type of solid support used, the type of system used for linking the GPCR polynucleotides to the solid support and the type of hybridisation chamber used; the characteristics of the biological polynucleotide test sample, such as purity, concentration, expected amount of cDNA, the quality of the cDNA etc. Depending on the before-mentioned factors, the hybridisation conditions may be adjusted to each individual array system. Depending on the said factors it is in general possible to use low stringent, medium stringent and high stringent hybridisation conditions. Preferably, high stringent conditions are used for human samples and medium stringent samples are used for rat and mouse samples. In connection with the present invention, an example of low stringent conditions is 40% formamide 1 M Na and a temperature of 370° C. An example of medium stringent conditions is 1 M Na and a temperature of 55° C. An example of high stringent conditions is 1 M Na and a temperature of 65° C. For all types of hybridisation, the incubation period is preferably more than 16 hours, more preferably more than 20 hours, and most preferably more than 24 hours.

According to one embodiment of the invention the GPCR array will be used for the evaluation of the expression profile of one or more biological materials or a mixture of biological materials. The method for the determination of a GPCR polynucleotide expression profile in a biological material or in a mixture of biological materials comprises obtaining a polynucleotide from the biological material(s), labelling said polynucleotide to obtain a labelled target polynucleotide sample, contacting at least one labelled target polynucleotide sample with an array as defined above under conditions which are sufficient to produce a hybridisation pattern and detecting said hybridisation pattern to obtain the GPCR polynucleotide expression profile of the biological material or the mixture of biological materials. The GPCR expression profile in the biological material can thus be determined to correspond to the expression of e.g. GPCR's such as adrenergic, adenosine, dopaminergic, histaminergic, opioid and serotonin GPCR's members, or any other polynucleotides encoding polypeptides capable of transducing a signal across biological membranes. The biological material or the mixture of biological materials may be in a non-stressed or a stressed stage. The stress may directly or indirectly influence the GPCR expression profile and thereby the polynucleotides identified which react upon that type of stress. The stress may be caused by a disease or a condition such as e.g. Asthma, cystic fibrosis, chronic obstructive pulmonary disease and rhinorrhea, convulsions, vascular spasms, coronary artery spasms, renal disorders, polycystic kidney disease, bladder spasms, urinary incontinence, bladder outflow obstruction, irritable bowel syndrome, gastrointestinal dysfunction, secretory diarrhoea, ischaemia, cerebral ischaemia, ischaemic hearth disease, angina pectoris, coronary hearth disease, traumatic brain injury, psychosis, anxiety, depression, dementia, memory and attention deficits, drug addiction and/or abuse, including cocaine or tobacco abuse, Parkinson's disease, Alzheimer's disease, dysmenorrhoea, narcolepsy, Reynaud's disease, intermittent claudication, Sjorgren's syndrome, migraine, arrhythmia, hypertension, absence seizures, myotonic muscle dystrophia, xerostomi, diabetes type II, hyperinsulinemia, premature labour, baldness, cancer, schizophrenia or psychosis.

A variety of disorders associated with the neural system, for example eating disorders, obsessive-compulsive disorders, panic disorders, alcoholism, pain, memory deficits and anxiety. Included among these disorders are disorders such as pseudodementia or Ganser's syndrome, migraine pain, bulimia, obesity, pre-menstrual syndrome or late luteal phase syndrome, post-traumatic syndrome, memory loss, memory dysfunction, social phobia, attention deficit hyperactivity disorder, chronic fatigue syndrome, premature ejaculation, erectile difficulty, anorexia nervosa, disorders of sleep, autism, mutism, trichotillomania or mood syndrome.

Auto-immune diseases, e.g. Addison's disease, alopecia areata, Ankylosing spondylitis, haemolytic anaemia (anaemia haemolytica), pernicious anaemia (anaemia pemiciosa), aphthae, aphthous stomatitis, arthritis, arteriosclerotic disorders, osteoarthritis, rheumatoid arthritis, aspermiogenese, asthma bronchiale, auto-immune asthma, auto-immune haemolysis, Becket's disease, Boeck's disease, inflammatory bowel disease, Burkett's lymphoma, Chron's disease, chorioiditis, colitis ulcerosa, Coeliac disease, cryoglobulinemia, dermatitis herpetiformis, dermatomyositis, insulin-dependant type I diabetes, juvenile diabetes, idiopathic diabetes insipidus, insulin-dependant diabetes mellisis, auto-immune demyelinating diseases, Dupuytren's contracture, encephalomyelitis, encephalomyelitis allergica, endophthalmia phacoanaphylactica, enteritis allergica, auto-immune enteropathy syndrome, erythema nodosum leprosum, idiopathic facial paralysis, chronic fatigue syndrome, febris rheumatica, glomerulo nephritis, Goodpasture's syndrome, Graves’ disease, Hamman-Rich's disease, Hashimoto's disease, Hashimoto's thyroiditis, sudden hearing loss, sensoneural hearing loss, hepatitis chronica, Hodgkin's disease, haemoglobinuria paroxysmatica, hypogonadism, ileits regionalis, iritis, leucopoenia, leukaemia, lupus erythematosus disseminatus, systemic lupus erythematosus, cutaneous lupus erythematosus, lymphogranuloma malignum, mononucleosis infectiosa, myasthenia gravis, traverse myelitis, primary idiopathic myxedema, nephrosis, ophthalmia symphatica, orchitis granulomatosa, pancreatitis, pemphigus, pemphigus vulgaris, polyarteritis nodosa, polyarthritis chronica primaria, polymyositis, polyradiculitis acuta, psoreasis, purpura, pyoderma gangrenosum, Quervain's thyreoiditis, Reiter's syndrome, sarcoidosis, ataxic sclerosis, progressive systemic sclerosis, scleritis, sclerodermia, multiple sclerosis, sclerosis disseminata, acquired spenic atrophy, infertility due to antispermatozoan antibodies, thrombocytopenia, idiopathic thrombocytopenia purpura, thymoma, acute anterior uveitis, vitiligo, AIDS, HIV, SCID and Epstein Barr virus associated diseases such as Sjorgren's syndrome, virus (AIDS or EBV) associated B cell lymphoma, parasitic diseases such as Lesihmania, and immune-suppressed disease states such as viral infections following allograft transplantations, graft vs. Host syndrome, transplant rejection, or AIDS, cancers, chronic active hepatitis diabetes, toxic chock syndrome, food poisoning, and transplant rejection.

These are examples and are not intended to limit the invention in any way.

The analysis of the expression profile includes several steps of procedures in which well known techniques are used, such as those mentioned in e.g. Sambrook et al., Molecular Cloning: A Laboratory approach, Cold Spring Harbour Press, NY (1987), and in Ausubel et al., Current Protocols in Molecular Biology, Greene Publishing Co. NY, (1995).

The biological material to be evaluated needs to be identified and isolated such as e.g. described in Example 2. For the ability to perform the analysis cDNA are generally produced from isolated total RNA or polyA RNA (mRNA). The total RNA/mRNA can be isolated using a variety of techniques. Numerous techniques are well known (see e.g. Sambrook et al. and Ausubel et al., op cit.). In general, these techniques include a first step of lysing the cells and then a second step of enriching for or purifying RNA.

The isolated total RNA/mRNA are reversed transcribed using a RNA-directed DNA polymerase, such as “reverse transcriptase” isolated from such retroviruses as AMV, MoMuLV or recombinantly produced. Many commercial sources are available (from e.g. Perkin Elmer, New England Biolabs, Stratagene Cloning Systems).

Preferably the mRNA is reversed transcribed into cDNA and at the same time a label is incorporated for later detection of the hybridised amplified products on the GPCR array. The amplification by PCR may be performed according to Example 2. The label may vary dependent on the system to be used for the detection and several labels are well known in the area of molecular biology (e.g. radioactive labels, fluorescent labels, colouring labels, chemical labels etc.)

The labelled cDNA is then denaturised and used for hybridisation on the GPCR array. The hybridisation conditions vary and are dependent on the aim with the expression profile obtained after the hybridisation. One example is found in Example 6. After hybridisation of the labelled cDNA, the GPCR array is washed to remove the cDNA, which have not hybridised to the GPCR and the hybridised labelled cDNA are detected by a suitable means and an expression profile obtained.

In a second embodiment of the present invention two GPCR arrays will be used for the evaluation of the expression profiles in at least a first and a second biological material. The expression profiles of the first and the second biological material are compared to each other to identify any differences between the first and the second expression profile. The analysis comprising obtaining a first GPCR expression profile of the first biological material as described above, obtaining a second GPCR expression profile of the second biological material as described for the first biological material, comparing the first and the second GPCR expression profile to identify any differences in the GPCR expression profiles between the first and the second GPCR expression profile. The first and the second biological material may be of the same origin of different origins, for example two livers from the same animal species or two lungs from the same animal or from two animals of the same species etc. In one embodiment of the invention, the first and the second biological material are in two different stages, i.e. the first biological material is non-stressed and the second biological material is stressed. The second biological material may be stressed in such as way that at least a different GPCR expression profile will be obtained. The stress may directly or indirectly influence the GPCR expression profile.

The GPCR's to be influenced by the stress of the second biological material may be any GPCR belonging to any of the overall group of Rhodopsin-like receptors, secretn-like, metabotropic glutamate/pheromone, Frizzled/smoothened and a large group of unclassified GPCR's. The groups of Rhodopsin-like receptors are typically divided into smaller subgroups like e. g. adrenergic, adenosinergic, dopaminergic, histaminergic, opioid, serotonergic and peptide hormone receptor like e.g. angiotensin, bradykinin, chemokine, endothelin and melanocortin, neuropeptide Y, somatostatin, tachykinin, galanin, orexin, rhodopsin, olfactory, prostaglandin, nucleotide-like and purinoceptor. Some of the GPCR belonging to the group of secretin-like receptorsion includes calcitonin, corticotropin releasing factor, gastric inhibitory peptide, glucagons, growth hormone-releasing hormone, parathyroid hormone, PACAP, secretin, vasoactive intestinal polypeptide, diuretic hormone, EMR1, latrotoxin. The metabotropic glutamate/pheromone GPCR's are exemplified by the metabotropic glutamate, extracellular calcium sensing, GABA-B and putative pheromone receptors.

Furthermore, the GPCR expression profile of the second biological material may be directly or indirectly related to a disease, chemical pre-treatment, environmental influences or other physiological or pathophysiological changes in the biological material. The chemical treatment may be selected from the group consisting. of naturally occurring chemical entities or synthetically derived chemical entities.

Examples of diseases or conditions that might influence the GPCR expression profile of the second biological material are those mentioned above. As an example the use of in vivo models such as e.g. a rat model in which at least a first and a second experimental group are used. The first group is non-stressed and the second group stressed in such a way that the expression of one or more GPCR polynucleotides are influenced in such as way that an increase or a decrease of the expression is obtained, when the expression profiles are analysed using the GPCR array and the method according to the invention. The second group may be either permanently stressed or stressed during a certain period of time and after the period of stress one or more biological materials obtained from the second group and the GPCR expression profile determined

In another embodiment of the present invention, the GPCR array is used for the evaluation of the expression profiles in at least a first and a second biological material, each material being labelled with a unique label (e.g. Cy3 and Cy5 for each sample, respectively). The procedure is as described above.

In a third embodiment according to the invention, the GPCR array will be used for the identification of a therapeutic, prophylactic or toxic agent involved in the response of GPCR polypeptides in a biological material, said method comprises obtaining a first GPCR expression profile of a first biological material as described above, obtaining a second GPCR expression profile of a second biological material as described above, treating the first and/or the second biological material with a test compound; obtaining a third and/or a fourth GPCR expression profile of the treated second biological material as described above, comparing the first, second, third and/or fourth GPCR expression profiles, and identifying any difference in the GPCR expression profile so as to identify any therapeutic, prophylactic or toxic response of the test compound on the GPCR polynucleotide. The first biological material may typically be a material in a healthy or normal condition whereas the second biological material typically may be in a diseased or not normal state. The first and the second biological material may have the same kind of origin.

The first biological material may be in a non-stressed state and the second biological material may be in a stressed state and the stress may directly or indirectly influence the GPCR expression profile between the first and the second biological materials. The GPCR polynucleotide family which is influenced by the stress is selected from the group consisting of voltage-gated GPCR's, Gap-junction GPCR's, ligand-gated GPCR's, heat-activated GPCR's, intracellular GPCR's, GPCR gated by intracellular ligands such as cyclic nucleotide-gated channels or calcium-activated GPCR's, and the GPCR expression profile of the second biological material is directly or indirectly related to a disease, a chemical or biological pre-treatment, environmental influences or other physiological or pathophysiological changes.

The disease may be anyone of those mentioned above.

The test compound may be a chemical or a biological compound including therapeutic, prophylactic and/or toxic chemical entities, physiologically chemical entities, substances affecting a biological function, hormones, vitamins, nutrients, pesticides, fungicides, bacteriocides and the like. The method according to the third embodiment of the invention is used to identify potential therapeutic, prophylactic and/or toxic agents useful for the treatment of diseases caused by an alteration in the expression profile of the GPCR polypeptides. One example is the use of a biological model, such as a rat model in which a first, second, third and/or fourth group are used. The first and third group is non-stressed and the second and fourth group stressed in such a way that the expression of one or more GPCR polynucleotides are influenced in such as way that an increase or a decrease of the expression is obtained. The third and fourth groups are treated with a test compound.

In a fourth embodiment the invention will be used in diagnostic methods to enable the determination in differences of GPCR expression profiles between two different biological material, said method comprises obtaining a first GPCR expression profile of a first biological material as described above, obtaining a second GPCR expression profile of a second biological material as described above, comparing the first and second GPCR expression profiles, and identifying any difference in the GPCR expression profile. Preferably a disease, such as chronic pain, myotonia, multiple sclerosis, and rheumatoid arthritis, directly or indirectly influences the difference between the GPCR expression profile of the first and the second GPCR expression profile. Furthermore, the GPCR expression profile from more than two different biological materials are compared, such as biological materials, which are in different stages of a disease. The diagnostic method may be useful in the determination of diseases directly or indirectly caused by different GPCR expression profiles and by the use of such a method there will be an enhanced possibility to start the treatment of the disease at an early stage of the disease.

GPCR Kits

The invention also relates to kits comprising the above-mentioned GPCR array.

The kit may be used according to the above-mentioned methods for the determination of GPCR expression profiles in a biological material as defined above.

The kit comprises the GPCR array as described above. However, the kit may further comprise reagents for generating a labelled target polynucleotide sample and/or a hybridisation buffer suitable for performing hybridisation between a biological material and the GPCR array.

### EXAMPLES

The invention is further illustrated with reference to the following examples, which are not intended to be in any way limiting to the scope of the invention as claimed.

### Example 1

Generation of Fragments for the GPCR Array (IC Array)

Regions of opioid receptor polynucleotides were identified using a method as described in the following in general terms.

For each GPCR family, all members are identified, and the cDNA sequence of the members were downloaded from Genbank and the sequences of the Open Reading Frames (ORF) were compared to the other family members by clustal alignment (Higgins, D. G., Sharp, P. M., Gene 1988 Dec. 15; 73(1); 237-244). For each family member in turn, the alignments were performed to identify non-conserved regions of the member in question having as low a sequence identity to the other members of the family as possible (intrafamily). The level of intrafamily identity varies from family to family and from family member to family member, but in general it is initially attempted to identify regions having a level of identity of below 60%. However, for some family members this is not possible, and in such cases regions having the lowest existing sequence identity are used. Subsequently, the regions identified on the basis of a low intrafamily sequence identity are compared to the corresponding GPCR family members from other species to determine the level of sequence identity. The level of interspecies identity varies from family to family and from family member to family member, but in general it is initially attempted to obtain regions having a level of identity of above 70%. If this condition is not met, a different region is selected on the basis of the intrafamily identity as described above, and the interspecies identity of the new region is determined. This procedure is repeated a number of times to optimise the region selected with respect to partly intrafamily identity and partly interspecies identity.

The opioid receptors belong to the GPCR gene family and is currently classified into three groups μ, δ and κ. It has been suggested that further subtypes like μ1 and μ2 exists but these will not be considered here.

The cDNA sequences corresponding to the p-opioid receptor (MOR), δ-opioid receptor (DOR) and κ-opioid receptor (KOR) from the rat (r) and human (h) species were downloaded from Genbank, and the sequences of the Open Reading Frames (ORF) were compared to each other by clustal alignment (see e.g. Higgins D G and Sharp P M, Gene 1988 73 (1) 237-244).

| Identity | GenBank No. |
|---|---|
| hMOR | NM_000914 |
| rMOR | NM_013071 |
| hDOR | NM_000911 |
| rDOR | NM_012617 |
| hKOR | XM_011716 |
| rKOR | L22536 |

The alignments were performed to identify non-conserved regions of the GPCR member, regions having less than 60% sequence homology to the other GPCR members and at the same time having more than 70% homology between identical GPCR members from rat and human. Two fragments A and B were selected which fulfils these criteria.

| | Fragment A - | Fragment B - |
|---|---|---|
| Identity | coding sequence | coding sequence |
| hMOR | 1-399 | 791-1203 |
| rMOR | 1-393 | 785-1197 |
| hDOR | 1-336 | 728-1119 |
| rDOR | 1-336 | 728-1119 |
| hKOR | 1-366 | 766-1143 |
| rKOR | 1-366 | 766-1143 |

Fragment A - percent identity (consensus length 406)

| Gene | hMOR | rMOR | hDOR | rDOR | hKOR | rKOR |
|---|---|---|---|---|---|---|
| hMOR | X | 80 | 48 | 45 | 55 | 51 |
| rMOR | | X | 44 | 43 | 57 | 55 |
| hDOR | | | X | 70 | 53 | 50 |
| rDOR | | | | X | 50 | 50 |
| hKOR | | | | | X | 72 |
| RKOR | | | | | | X |

Fragment B - percent identity (consensus length 420)

| Gene | hMOR | rMOR | hDOR | rDOR | hKOR | rKOR |
|---|---|---|---|---|---|---|
| hMOR | X | 78 | 45 | 47 | 50 | 49 |
| rMOR | | X | 49 | 51 | 51 | 53 |
| hDOR | | | X | 83 | 47 | 47 |
| rDOR | | | | X | 49 | 47 |
| hKOR | | | | | X | 72 |
| RKOR | | | | | | X |

Fragment A has a maximal of 57% identity between two intrafamily members (compare hKOR and rMOR) while the minimum interspecies identity is 70% (compare hDOR and rDOR). Thus the minimum difference in percentage intrafamily and interspecies identity is 13% which is sufficient to avoid cross hybridisation between intrafamily members and a the same time get signal from interspecies members.

Fragment B has a maximal of 53% identity between two intrafamily members (compare rMOR and rKOR) while the minimum interspecies identity is 72% (compare hKOR and rKOR). Thus the minimum difference in percentage intrafamily and interspecies identity is 19% which is sufficient to avoid cross hybridisation between intrafamily members and a the same time get signal from interspecies members.

### Example 2

Amplification of the Determined Regions

The two regions A (consensus sequence: 1-406) and B (consensus sequence: 801-1216) were amplified by PCR using following primers:

| | | |
|---|---|---|
| Region | Primer 1 | Primer 2 |

| | | |
|---|---|---|
| hMOR-fragment | 5′ ATG GAC AGC AGC | 5′ TCC CAT TAG GTA |
| A | GCT GCC C | ATT CAC AC |
| hMOR-fragment | 5′ TCC GCA TGC TCT | 5′ TTA GGG CAA CGG |
| B | CTG GCT GC | AGC AGT TTC |
| hDOR-fragment | 5′ ATG GAA CCG GCC | 5′ CTC CAT CAG GTA |
| A | CCC TCC | CTT GGC AC |
| hDOR-fragment | 5′ TGC GCC TGC TGT | 5′ TCA GGC GGC ACG |
| B | CGG GCT C | GCC ACC |
| hKOR-fragment | 5′ ATG GAC TCC CCG | 5′ ATT CAT CAA GTA |
| A | ATC CAG | GAC CGT AC |
| hKOR-fragment | 5′ TCC GGC TCC TTT | 5′ TCA TAC TGG TTT |
| B | CTG GCT C | ATT CAT CC |

Human whole brain mRNA was purchased from Clontech Laboratories Cat. No. 6516-1.

The cDNA was synthesized using the Omniscript RT Kit (Qiagen, 205111).

The amplification of the two regions were performed using:

1-2 μl cDNA obtained above

250 μM dNTP (27-2035-1, Amersham Pharmacia)

0.5 μM of each PCR primers (see table above)

1.5 μM MgCl₂ final concentration (Y02016, Gibco BRL)

1× PCR buffer without MgCl₂ (Y02028, Gibco BRL)

2.5 U Taq polymerase (10966-026, Gibco BRL)

H₂O to 20 μl final volume

Using PCR conditions according to the supplier's manual.

The PCR generated fragments were separated using on a conventional agarose gel and cloned into the pCRII-TOPO vector according to the TOPO TA cloning kit (Invitrogen) and the nucleotide sequence was analysed using CEQ 2000 DNA Analysis System (Beckman Coulter, U.S.A.).

### Example 3

Preparation of Master Glycerol Stocks

The glycerol stocks were prepared in 96 wells-trays (Corning Cat. No. cci3793) on a Biomek (Beckman Coulter, USA). 50 μl glycerol media was transferred into each well of a plane 96-well tray (Corning Cat. No. cci3793). 50 μl bacterial culture was transferred into each well of the plane 96-well tray and mixed with 2×100 μl. A Storage Mat-l lid (Corning, Cat. No. 3094) was placed on each tray and the trays stored at −80° C.

### Example 4

Preparation of Plasmids for the GPCR Array

Ampicillin (100 mg/ml) was added to Circlegrow medium (obtained from Bio101, Carlsbad, Calif. 92008, U.S.A.). Circlegrow medium/ampicillin was added to each well in a 4×2 ml 96 deep-well tray (Corning Cat. No. cci 3961). The glycerol stocks were added to each well. The tray was sealed with sealing sheet (Merck Eurolab A/S, Denmark), and incubated with shaking at 37° C. for 16 hours prior to plasmid purification.

### Example 5

Preparation of 3D-Link Amine-Binding Array

The plasmids obtained in Example 4 were subjected to PCR using flanking primers. The resulting PCR product was spotted onto a 3D-Link Amine-Binding slide (array). The PCR reaction and spotting were carried out using standard methods as described e.g. in “Microarray Biochip Technology” by Mark Schena and “DNA Microarrays: A Practical Approach” by Mark Schena.

3D-Link Amine-Binding slide (array) was obtained from SurModics, Inc, Minneapolis, USA.

To have an orientation on the arrays visible after scanning of the slides the each of the arrays comers are marked by a double labelled Cy3 and Cy5 primer with a 5′ end amino group (5′-Cy3/5) in a final concentration of 1 pmol/μl to enable the possibility to place a grid on the scanned array.

After spotting, the slide is sealed with sealing tape and stored at −20° C. until use.

3 MM paper is pre-wetted with saturated NaCl solution. All slides are places in a slide box without a lid, and the slide boxes are placed in a plastic bag containing the NaCl saturated 3 MM paper. The plastic bag is closed and the slides are incubated.

After incubation the slides are removed from the plastic bag and stored at room temperature.

The slides are placed in pre-heated blocking solution (0.1% SDS, SurModics Blocking Solution), incubated for 20 minutes at 50° C., and washed in redistilled H₂O. The slides are incubated in 4×SSC, 0.1% SDS solution (50° C.), washed at room temperate in redistilled H₂O. The slides are incubated in boiling redistilled H₂O for 2 minutes, and washed in redistilled H₂O at room temperature. The slides are incubated in pre-hybridisation buffer pre-heated to 50° C. (50 ml 20×SSC, 10 ml 100×Denhardt solution, 2 ml 10% SDS, 4 ml salmon sperm DNA (10 mg/ml), 134 ml Redistilled H₂O) at 50° C. for 30 minutes. The slides are washed in redistilled H₂O, and stored at room temperature in a dry and dark place until further use.

### Example 6

Preparation of Labelled Samples and Hybridisation

Whole human brain total RNA is obtained from Clontech Laboratories Cat. No. 64020-1). RNA is precipitated by centrifugation and the RNA pellet is washed in 70% ethanol. RNA is precipitated at 15,000×g for 15 minutes. The supernatant is discarded and the pellet let to air-dry. The RNA concentration is adjusted to 1 μg/μl with DEPC-H₂O. In 2 separate tubes 25 μl total RNA (1 μg/μl) and 7 μl DEPC treated H₂O are added. 4 μl of oligo-dT (e.g. T25V primer) (1 μg/μl) are added to each tube. The tubes are incubated in a Thermal cycler at 65° C. for 3 minutes.

Tube 1 is prepared by adding, 5 μl 110×cDNA Buffer (500 mM Tris-HCl, pH 8.3; 800 mM KCl; 100 mM MgCl₂; 40 mM DTT), 2 μl Cy3-dUTP (1 mM, Amersham Pharmabiotech Cat. No. PA53022), 5 μl 10×dNTP (5 mM dATP; 5 mM dCTP; 5 mM dGTP; 5 mM dTTP). The contents are mixed and 2 μl reverse transcriptase (100 U/μl) are added.

Tube 2 is prepared by adding, 5 μl 110×cDNA Buffer (500 mM Tris-HCl, pH 8.3; 800 mM KCl; 100 mM MgC₂; 40 mM DTT), 2 μl Cy5-dUTP (1 mM, Amersham Pharmabiotech Cat. No. PA55022), 5 μl 10×dNTP (5 mM dATP; 5 mM dCTP; 5 mM dGTP; 5 mM dTTP). The contents are mixed and 2 μl reverse transcriptase (100 U/μl) are added.

Tubes 1 and 2 are incubated at 42° C. for 60 minutes, and at 65° C. for 15 minutes. The temperature is decreased to 42° C. Reverse transcriptase (100 ∪/μl) is added to each tube followed by incubation at 42° C. for 60 minutes and at 65° C. for 15 minutes. Precipitate with 3M Na-acetate and 96% ethanol. Wash each pellet in 80% ethanol.

Each pellet is resuspended in RNase Mix (10 mM Tris-HCl (pH 7.5), 0.1 mM EDTA (pH 8.0), RNase A 100 mμ/ml) and incubated at 37° C. for 60 minutes. 30 μl of sterile H₂O are added to each tube. Precipitation is accomplished using 3M Na-Acetate (pH 6.0) and ice-cold 96% ethanol. The pellets are washed in 80% ethanol, and resuspended in 15 μl hybridisation buffer (5×SSC, 0.1% SDS, 100 μg/ml, blocking RNA).

The two fluorescents probes are mixed 1:1 in a PCR tube. This is the Probe-Mix.

The Probe-Mix is denatured at 100° C. for 3 minutes, followed by a temperature decrease to 55° C. for 30 seconds, where after the Probe-Mix is placed on ice. The Probe-Mix is added to the array slide, and the slides are placed in a box and inside the petri dish with the pre-wetted 3 MM paper. The lid back is replaced onto the petri dish, and the petri dish is placed in a plastic bag.

The petri dish is incubated in a dark incubator at 65° C. 12-16 hours.

The slides are washed in Washing Buffer I (2×SSC), and submerged in pre-warmed Washing Buffer II (2×SSC, 0.1% SDS). The slides are pre-heated at 65° C. for 1 hour in a volume of least 10 ml/slide to cover the slides, and incubated on an orbital shaker at 65° C. for 10 minutes. The slides are washed in Washing Buffer III (0.2×SSC) in a volume of least 10 ml/slide to cover the slides, and incubated on an orbital shaker at room temperature for 3 minutes. The slides are washed in Washing Buffer IV (0.1×SSC), and in Washing Buffer V (0.5×SSC). Washing with Washing Buffer V is repeated for additional 3 times, and all Washing Buffer V is removed by centrifuging at 800 rpm for 3 minutes.

Scanning of the slides and evaluation are performed using an Affymetrix 418 Scanner, Affymetrix 418 Scanner Software and ImaGene 4.0 software (BioDiscovery) according to the supplier's manual.

1. A GPCR array comprising a multiplicity of individual GPCR polynucleotide spots stably associated with a surface of a solid support, wherein an individual GPCR polynucleotide spot comprises a GPCR polynucleotide composition comprising a non-conserved region of a GPCR polynucleotide family member, the spots representing at least two different regions of a GPCR polynucleotide family member.

2. The array according to claim 1, wherein the multiplicity of individual spots represents at least two different GPCR polynucleotide family members and/or at least two different GPCR families.

3. The array according to any of the preceding claims, wherein the non-conserved regions of the GPCR polynucleotide family member has less than 90% intrafamily identity.

4. The array according to any of the preceding claims, wherein the non-conserved regions of the GPCR polynucleotide family member has less than 85% intrafamily identity.

5. The array according to any of the preceding claims, wherein the non-conserved regions of the GPCR polynucleotide family member has less than 80% intrafamily identity.

6. The array according to any of the preceding claims, wherein the non-conserved regions of the GPCR polynucleotide family member has less than 75% intrafamily identity.

7. The array according to any of the preceding claims, wherein the non-conserved regions of the GPCR polynucleotide family member has less than 50% intrafamily identity.

8. The array according to any of the preceding claims, wherein the non-conserved regions of the GPCR polynucleotide family member has at least 50% interspecies identity.

9. The array according to claim 8, wherein the non-conserved regions of the GPCR family member has at least 60% interspecies identity.

10. The array according to claim 9, wherein the non-conserved regions of the GPCR family member has at least 65% interspecies identity.

11. The array according to claim 10, wherein the non-conserved regions of the GPCR family member has at least 70% interspecies identity.

12. The array according to claim 11, wherein the non-conserved regions of the GPCR family member has at least 75% interspecies identity.

13. The array according to claim 12, wherein the non-conserved regions of the GPCR family member has at least 80% interspecies identity.

14. The array according to any of the preceding claims, wherein the non-conserved regions of the GPCR polynucleotide family member has an average length of from about 3 to about 9,000 nucleotides, from about 3 to about 6,000, from about 3 to about 3,000, from about 200 to 750, from about 200 to 700, from about 200 to 500, from about 250 to 400 or from about 290 to 350.

15. The array according to any of the preceding claims, wherein said different regions of a GPCR polynucleotide family member are polynucleotide regions from the same polynucleotide strand and the regions are at least non-overlapping polynucleotide regions of the strand.

16. The array according to any of the preceding claims, wherein a GPCR polynucleotide family is polynucleotides encoding polypeptides capable of transducing a signal across biological membranes.

17. The array according to any of the preceding claims, wherein the GPCR polynucleotide family is selected from the group consisting of GPCR's belonging to any of the overall groups of Rhodopsin-like receptors, secretin-like, metabotropic glutamate/pheromone, Frizzled/smoothened and a large groups of unclassified GPCR's, which are all characterized by they seven transmembrane spanning regions and their ability to transduce a signal across a cellular membrane.

18. The array according to any of the preceding claims, wherein the polynucleotide composition comprises one or more of the same non-conserved region of a GPCR polynucleotide family member.

19. The array according to claim 18, wherein the polynucleotide composition comprises one or more of the same non-conserved region in the single stranded or double stranded form.

20. The array according to any of the preceding claims, wherein the non-conserved region of a GPCR polynucleotide family member is of DNA, RNA, cDNA, natural, synthetic, semi-synthetic origin or is a chemical analogous such as LNA and PNA.

21. The array according to any of the preceding claims, wherein the non-conserved region of a GPCR polynucleotide family member is obtained from one or more biological materials such as e.g. an organism, an organ, a tissue, a cell or a biological material produced by a cell culture.

22. The array according to claim 21, wherein the biological material is an organism, such as a microorganism, a plant, a fungus or an animal.

23. The array according to claim 22, wherein the biological material is an animal.

24. The array according to claim 23, wherein the animal is selected from the group consisting of humans, rats, mice, pigs, cows, horses, dogs, guinea pigs, ferrets, rabbits, apes, monkeys, cats and sheep.

25. The array according to any of the preceding claims, wherein the solid support is made of a flexible or rigid material.

26. The array according to any of the preceding claims, wherein said array comprises from about 2 to about 100,000 such as, e.g. from about 2 to about 50,000, from about 10 to about 25,000, from about 100 to about 10,000, from about 100 to about 5,000, from about 100 to about 1,000, from about 400 to about 600 or about 500 GPCR polynucleotide spots, or at least 2 such as, e.g. at least 10, at least 25, at least 50, at least 100, at least 300, at least 400, at least 500 or at least 600 spots.

27. A method of preparing an array according to any of the preceding claims, said method comprising a) generating said non-conserved regions of GPCR polynucleotide family members, b) preparing a multiplicity of compositions each comprising at least a non-conserved region, and c) stably associating said compositions in individual spots on a surface of a solid support.

28. The method according to claim 27, wherein each said non-conserved region of a GPCR polynucleotide family member is produced by one or more primers specific for said non-conserved region.

29. A set of primers specific for non-conserved regions of GPCR polynucleotide family members, wherein the set of primers are used in the method according to any of the claims 27-28 for the production of an array according to any of the claims 1-26.

30. A method for the determination of a GPCR polynucleotide expression profile in a biological material, said method comprising a) obtaining a polynucleotide sample from the biological material, b) labelling said sample to obtain a labelled target polynucleotide sample, c) contacting at least one labelled target polynucleotide sample with an array according to any of the claims 1-25 under conditions which are sufficient to produce a hybridisation pattern, and d) detecting said hybridisation pattern to obtain the GPCR polynucleotide expression profile of the biological material.

31. A method for the determination of a difference in GPCR polynucleotide expression profiles from at least a first and a second different biological material, said method comprising obtaining a first GPCR expression profile of the first biological material according to the method of claim 29, obtaining a second GPCR expression profile of the second biological material according to the method of claim 29, comparing the first and the second GPCR expression profiles to identify any difference in the GPCR expression profiles between the first and the second GPCR expression profiles.

32. The method according to any of the claims 30-31, wherein the first and the second biological material is of the same kind of biological material.

33. The method according to the claims 30-32, wherein the first biological material is in a non-stressed state and the second biological material is in a stressed state.

34. The method according to the claim 32, wherein the stress directly or indirectly influence the GPCR expression profile of the first and/or the second biological material.

35. The method according to any of the claims 30-34, wherein the GPCR polynucleotide family is selected from the group consisting of GPCR's belonging to any of the overall groups of Rhodopsin-like receptors, secretin-like, metabotropic glutamate/pheromone, Frizzled/smoothened and a large groups of unclassified GPCR's, which are all characterized by they seven transmembrane spanning regions and their ability to transduce a signal across a cellular membrane.

36. The method according to claim 35, wherein the GPCR expression profile of the second biological sample is directly or indirectly related to a disease, chemical treatment, biological sample or parts in a biological sample treatment, environmental influences or other physiological or pathophysiological changes.

37. The method according to claim 36, wherein the chemical treatment is selected from the group consisting of naturally occurring chemical entities or synthetically derived chemical entities.

38. The method according to claim 36, wherein the disease is selected from the group consisting of asthma, cystic fibrosis, chronic obstructive pulmonary disease and rhinorrhea, convulsions, vascular spasms, coronary artery spasms, renal disorders, polycystic kidney disease, bladder spasms, urinary incontinence, bladder outflow obstruction, irritable bowel syndrome, gastrointestinal dysfunction, secretory diarrhoea, ischaemia, cerebral ischaemia, ischaemic hearth disease, angina pectoris, coronary hearth disease, traumatic brain injury, psychosis, anxiety, depression, dementia, memory and attention deficits, drug addiction and/or abuse, including cocaine or tobacco abuse, Parkinson's disease, Alzheimer's disease, dysmenorrhoea, narcolepsy, Reynaud's disease, intermittent claudication, Sjorgren's syndrome, migraine, arrhythmia, hypertension, absence seizures, myotonic muscle dystrophia, xerostomi, diabetes type II, hyperinsulinemia, premature labour, baldness, cancer, schizophrenia or psychosis; a variety of disorders associated with the neural system, for example eating disorders, obsessive compulsive disorders, panic disorders, alcoholism, pain, memory deficits and anxiety including disorders such as pseudodementia or Ganser's syndrome, migraine pain, bulimia, obesity, pre-menstrual syndrome or late luteal phase syndrome, post-traumatic syndrome, memory loss, memory dysfunction, social phobia, attention deficit hyperactivity disorder, chronic fatigue syndrome, premature ejaculation, erectile difficulty, anorexia nervosa, disorders of sleep, autism, mutism, trichotilomania or mood syndrome; auto-immune diseases, e.g. Addison's disease, alopecia areata, Ankylosing spondylitis, haemolytic anaemia (anaemia haemolytica), pernicious anaemia (anaemia perniciosa), aphthae, aphthous stomatitis, arthrits,. arteriosclerotic disorders, osteoarthritis, rheumatoid arthritis, aspermiogenese, asthma bronchiale, auto-immune asthma, auto-immune haemolysis, Becket's disease, Boeck's disease, inflammatory bowel disease, Burkett's lymphoma, Chron's disease, chorioiditis, colitis ulcerosa, Coeliac disease, cryoglobulinemia, dermatitis herpetiformis, dermatomyosits, insulin-dependent type I diabetes, juvenile diabetes, idiopathic diabetes insipidus, insulin-dependent diabetes mellisis, auto-immune demyelinating diseases, Dupuytren's contracture, encephalomyelitis, encephalomyelitis allergica, endophthalmia phacoanaphylactica, enteritis allergica, auto-immune enteropathy syndrome, erythema nodosum leprosum, idiopathic facial paralysis, chronic fatigue syndrome, febris rheumatica, glomerulo nephritis, Goodpasture's syndrome, Graves'disease, Hamman-Rich's disease, Hashimoto's disease, Hashimoto's thyroiditis, sudden hearing loss, sensoneural hearing loss, hepatitis chronica, Hodgkin's disease, haemoglobinuria paroxysmatica, hypogonadism, ileitis regionalis, iritis, leucopoenia, leukaemia, lupus erythematosus disseminatus, systemic lupus erythematosus, cutaneous lupus erythematosus, lymphogranuloma malignum, mononucleosis infectiosa, myasthenia gravis, traverse myelitis, primary idiopathic myxedema, nephrosis, ophthalmia symphatica, orchitis granulomatosa, pancreattis, pemphigus, pemphigus vulgaris, polyarteritis nodosa, polyarthritis chronica primaria, polymyositis, polyradiculitis acuta, psoreasis, purpura, pyoderma gangrenosum, Quervain's thyreoiditis, Reiter's syndrome, sarcoidosis, ataxic sclerosis, progressive systemic sclerosis, scleritis, sclerodermia, multiple sclerosis, sclerosis disseminata, acquired spenic atrophy, infertility due to antispermatozoan antibodies, thrombocytopenia, idiopathic thrombocytopenia purpura, thymoma, acute anterior uveitis, vitiligo, AIDS, HIV, SClD and Epstein Barr virus associated diseases such as Sjorgren's syndrome, virus (AIDS or EBV) associated B cell lymphoma, parasitic diseases such as Lesihmania, and immune-suppressed disease states such as viral infections following allograft transplantations, graft vs. Host syndrome, transplant rejection, or AIDS, cancers, chronic active hepatitis diabetes, toxic chock syndrome, food poisoning, and transplant rejection.

39. A method for identifying a therapeutic, prophylactic and/or toxic agent involved in a direct or indirect action on the GPCR expression profile in a biological material, said method comprises obtaining a first GPCR expression profile of a first biological material according to the method of claim 30, obtaining a second GPCR expression profile of a second biological material according to the method of claim 29, applying a test compound to the second biological material and obtaining a third GPCR expression profile thereof according to the method of claim 29, comparing the first, second and third GPCR expression profiles, and identifying any differences in the GPCR expression profiles so as to identify any biological response of the test compound on the GPCR expression profile.

40. The method according to claim 39 further comprising applying a test compound to the first biological material and obtaining a fourth GPCR expression profile thereof according to the method of claim 30, comparing the first, second, third and fourth GPCR expression profiles, and identifying any differences in the GPCR expression profiles so as to identify any biological response of the test compound on the GPCR expression profile.

41. The method according to claims 39 or 40, wherein the first and the second biological material is of the same kind of biological material.

42. The method according to the claims 38-41, wherein the first biological material is in a non-stressed state and the second biological material is in a stressed state.

43. The method according to the claim 42, wherein the stress directly or indirectly influence the GPCR expression profile of the first and/or the second biological material.

44. The method according to any of the claims 39-43, wherein the GPCR polynucleotide family is selected from the group consisting of GPCR's belonging to any of the overall groups of Rhodopsin-like receptors, secretin-like, metabotropic glutamate/pheromone, Frizzled/smoothened and a large groups of unclassified GPCR's, which are all characterized by they seven transmembrane spanning regions and their ability to transduce a signal across a cellular membrane.

45. The method according to claim 44, wherein the GPCR expression profile of the second biological material is direct or indirect measure of a diseased state, a chemical pre-treatment, or environmental influences or other physiological or pathophysiological changes.

46. The method according to claim 45, wherein the disease is selected from the same group as defined in claim 38.

47. The method according to any of the claims 39-46, wherein the test compound is a chemical or biological derived compound such as compounds selected from the group consisting of therapeutic, prophylactic and/or toxic chemical entities, physiologically chemical entities, hormones, vitamins, nutrients, pesticides, fungicides, bateriocides and any other organic chemical entity.

48. The method according to any of the claims 30-47, wherein about 100 μg or less of total RNA of the biological material is used for hybridisation on each individual GPCR polynucleotide spot.

49. A diagnostic method to determine the differences of GPCR expression profiles between two biological materials; said method comprises obtaining a first GPCR expression profile of a first biological material according to the method of claim 30, obtaining a second GPCR expression profile of a second biological material according to the method of claim 30, comparing the first and second GPCR expression profile, and identifying any difference in the GPCR expression profiles.

50. The diagnostic method according to claim 49, wherein the difference between the GPCR expression profile of the first and the second GPCR expression profile is directly or indirectly influenced by a pathophysiological state or a disease such as diseases claimed in claim 38.

51. The diagnostic method according to claim 50, wherein the GPCR expression profile from more than two different biological materials are compared, such as biological materials, which are in different stages of a disease.

52. The method according to any of the claims 30-51, wherein the biological material is an organism, such as a microorganism, a plant, a fungus or an animal.

53. The method according to claim 52, wherein the animal is selected from the group consisting of humans, rats, mice, pigs, cows, horses, dogs, guinea pigs, ferrets, rabbits, apes, monkeys, cats and sheep.

54. A GPCR kit for use in a hybridisation assay, said kit comprising a GPCR array according to any of claims 1-26.

55. The GPCR kit according to claim 54, wherein said kit further comprises reagents for generating a labelled target polynucleotide sample.

56. The GPCR kit according to the claims 54-55, wherein said kit further comprises a hybridisation buffer.
